SEQUENCE LISTING (26 sequences)

SEQ ID NO: 1 - protein, 25 aa, Artificial Sequence
  synthetic rice/Arabidopsis non-expresser of pathogenesis-related (PR) genes 1 (NPR1, NIM1, SAI1)-binding consensus sequence
XVXXVXXXEVXEFXXILRRXXXATR

SEQ ID NO: 2 - protein, 25 aa, Artificial Sequence
  synthetic rice/tobacco non-expresser of pathogenesis-related (PR) genes 1 (NPR1, NIM1, SAI1)-binding consensus sequence
XXXXXSXAEVXEFXAILRRMXXAXR

SEQ ID NO: 3 - protein, 27 aa, Artificial Sequence
  synthetic rice/soybean non-expresser of pathogenesis-related (PR) genes 1 (NPR1, NIM1, SAI1)-binding consensus sequence
XXXXXXXXEVEEFXAILRRMRXAXXTR

SEQ ID NO: 4 - protein, 25 aa, Artificial Sequence
  synthetic rice/wheat non-expresser of pathogenesis-related (PR) genes 1 (NPR1, NIM1, SAI1)-binding consensus sequence
XVDXVSDAEVEEFYAILRRMRDAXR

SEQ ID NO: 5 - protein, 25 aa, Artificial Sequence
  synthetic Arabidopsis non-expresser of pathogenesis-related (PR) genes 1 (NPR1, NIM1, SAI1)-binding consensus sequence
VVRTVTEEEVDEFFKILRRVHVATR

SEQ ID NO: 6 - protein, 24 aa, Artificial Sequence
  synthetic rice non-expresser of pathogenesis-related (PR)genes 1 (NPR1, NIM1, SAI1)- binding consensus sequence
VDEVSDAEVEEFYAILRRMRDATR

SEQ ID NO: 7 - protein, 25 aa, Artificial Sequence
  synthetic tobacco non-expresser of pathogenesis-related (PR) genes 1 (NPR1, NIM1, SAI1)- binding consensus sequence
VSPPPSEAEVDEFFAILRRMHVAVR

SEQ ID NO: 8 - protein, 24 aa, Artificial Sequence
  synthetic wheat non-expresser of pathogenesis-related (PR) genes 1 (NPR1, NIM1, SAI1)-binding consensus sequence
VDDVSDAEVEEFYAILRRMRDASR

SEQ ID NO: 9 - protein, 7 aa, Artificial Sequence
  synthetic negative regulator of disease resisitance (NRR)C-terminal ethylene responsive factor (ERF)-associated amphiphilic repression (EAR) motif-like sequence
LDLNXXP

SEQ ID NO: 10 - protein, 131 aa, Oryza sativa
  rice negative regulator of disease resisitance (NRR)
MDATTTDATTAKRKRPAASDIADDAPTTVDEVSDAEVEEFYAILRRMRDATRRLGARPPPPRAPAWRPSFSWEDFADAPPKQAPPPPQQPADHERVAENATPPRRPAPGLDLNVEPPSDAPATPRSARAPA

SEQ ID NO: 11 - protein, 135 aa, Triticum sp.
  synthetic wheat negative regulator of disease resisitance (NRR)
MDAPSPTAKRKRSSSATAAPDPVGVDDVSDAEVEEFYAILRRMRDASRRLVSGGVAAATARATPAPRAPAWCPSFSWEDFAPPAPTPPPPSQQQQQQREQLLPADERVAENASPPRRPVPRGLDLNAEPEPEVQA

SEQ ID NO: 12 - protein, 122 aa, Arabidopsis thaliana
  synthetic thale cress non-expresser of pathogenesis-related (PR) genes 1 (NIM1/NPR1) interacting protein 2 (NIMIN2)
MNNSLKKEERVEEDNGKSDGNRGKPSTEVVRTVTEEEVDEFFKILRRVHVATRTVAKVNGGVAEGELPSKKRKRSQNLGLRNSLDCNGVRDGEFDEINRVGLQGLGLDLNCKPEPDSVSLSL

SEQ ID NO: 13 - protein, 85 aa, Nicotiana tabacum
  tobacco G8-1
MDGEKKRKRTENGKANGGDRNRHERKSAANEHTAVSPPPSEAEVDEFFAILRRMHVAVRYLQESGQKRVVPKGDLDLNTLPGNGD

SEQ ID NO: 14 - protein, 113 aa, Artificial Sequence
  synthetic rice negative regulator of disease resisitance (NRR) amino acids 10-122
TAKRKRPAASDIADDAPTTVDEVSDAEVEEFYAILRRMRDATRRLGARPPPPRAPAWRPSFSWEDFADAPPKQAPPPPQQPADHERVAENATPPRRPAPGLDLNVEPPSDAPA

SEQ ID NO: 15 - protein, 129 aa, Artificial Sequence
  synthetic wheat negative regulator of disease resisitance (NRR) amino acids 7-135
TAKRKRSSSATAAPDPVGVDDVSDAEVEEFYAILRRMRDASRRLVSGGVAAATARATPAPRAPAWCPSFSWEDFAPPAPTPPPPSQQQQQQREQLLPADERVAENASPPRRPVPRGLDLNAEPEPEVQA

SEQ ID NO: 16 - protein, 27 aa, Artificial Sequence
  synthetic rice negative regulator of disease resisitance (NRR) NPR1-binding domain, amino acids 26-52
PTTVDEVSDAEVEEFYAILRRMRDATR

SEQ ID NO: 17 - protein, 29 aa, Artificial Sequence
  synthetic rice/Arabidopsis negative regulator of disease resisitance (NRR) NPR1-binding domain consensus sequence
PXTXXVXXVXXXEVXEFXXILRRXXXATR

SEQ ID NO: 18 - protein, 29 aa, Artificial Sequence
  synthetic Arabidopsis negative regulator of disease resisitance (NRR) NPR1-binding domain, amino acids 25-53
PSTEVVRTVTEEEVDEFFKILRRVHVATR

SEQ ID NO: 19 - protein, 33 aa, Artificial Sequence
  synthetic Arabidopsis negative regulator of disease resisitance (NRR) NPR1-binding domain, amino acids 22-54
ADDAPTTVDEVSDAEVEEFYAILRRMRDATRRL

SEQ ID NO: 20 - protein, 33 aa, Artificial Sequence
  synthetic rice/tobacco negative regulator of disease resisitance (NRR) NPR1-binding domain consensus sequence
ABXXXXXXXXXSXAEVXEFXAILRRMXXAXRXL

SEQ ID NO: 21 - protein, 33 aa, Artificial Sequence
  synthetic tobacco negative regulator of disease resisitance (NRR) NPR1-binding domain, amino acids 29-61
ANEHTAVSPPPSEAEVDEFFAILRRMHVAVRYL

SEQ ID NO: 22 - protein, 33 aa, Artificial Sequence
  synthetic rice negative regulator of disease resisitance (NRR) NPR1-binding domain, amino acids 20-52
DIADDAPTTVDEVSDAEVEEFYAILRRMRDATR

SEQ ID NO: 23 - protein, 33 aa, Artificial Sequence
  synthetic rice/soybean negative regulator of disease resisitance (NRR) NPR1-binding domain consensus sequence
DXXDXXPTXXXXXXXXEVEEFXAILRRMRXAXX

SEQ ID NO: 24 - protein, 27 aa, Artificial Sequence
  synthetic soybean (Glycine max) negative regulator of disease resisitance (NRR) NPR1-binding domain, aminoacids 145-225
DGSDGVPTEEEVEEFFAILRRMRMAVK

SEQ ID NO: 25 - DNA, 27 bp, Artificial Sequence
  synthetic PCR amplification primer NRRC-pET for C-terminal of NRR (NRRC)
tttcatatggacgccacccgacggctc

SEQ ID NO: 26 - DNA, 35 bp, Artificial Sequence
  synthetic PCR amplification primer mn45-4 for C-terminal of NRR (NRRC)
aggatccactagtctcgagttgtaatccgtgagca

(19) United States
(12) Patent Application Publication
(10) Pub. No.: US 2010/0037349 A1
(43) Pub. Date: Feb. 11, 2010

(54) VARIANTS OF NRR ACTIVATE PLANT DISEASE RESISTANCE

(75) Inventors: Mawsheng Chern, Davis, CA (US); Pamela Ronald, Davis, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: REGENTS OF THE UNIVERSITY OF CALIFORNIA, Oakland, CA (US)

(21) Appl. No.: 12/417,315

(22) Filed: Apr. 2, 2009

Related U.S. Application Data
(60) Provisional application No. 61/123,007, filed on Apr. 4, 2008.

(51) Int. Cl. A01H 5/00; C12N 15/82; C07H 21/04; C12N 15/63; C12N 5/10

(52) U.S. Cl. 800/279; 800/301; 536/23.6; 435/320.1; 435/419

(57) ABSTRACT

Methods and compositions for improving plant disease resistance by expression of NPR1-biding domain/transcriptional activation domain fusions are provided.

### CROSS-REFERENCE TO RELATED PATENT APPLICATIONS

The present patent application claims benefit of priority to U.S. Provisional Patent Application No. 61/123,007, filed Apr. 4, 2008, which is incorporated by reference for all purposes.

### STATEMENT AS TO RIGHTS TO INVENTIONS MADE UNDER FEDERALLY SPONSORED RESEARCH AND DEVELOPMENT

This invention was made with Government support under US Department of Agriculture grant number 2004-63560416640 and under National Science Foundation grant number 0096901. The Government has certain rights in this invention.

### BACKGROUND OF THE INVENTION

Systemic acquired resistance (SAR) is an induced defense response in plants; it induces expression of pathogenesis-related (PR) genes Ryals, J. A. et al., Plant Cell 8:1809-1819 (1996) and confers lasting broad-spectrum resistance to viral, bacterial, and fungal pathogens. In dicots, such as Arabidopsis and tobacco, the phytohormone salicylic acid (SA) and the synthetic chemicals 2,6-dichloroisonicotinic acid (INA) and benzothiadiazole (BTH) are potent inducers of SAR (Friedrich, L. et al., Plant J. 9:61-70 (1996)). The NPR1 (for non-expresser of PR genes 1; also known as NIM1 and SAI1) gene is identified as a key regulator of the SA-mediated SAR pathway in Arabidopsis (Cao, H. et al., Plant Cell 6:1583-1592 (1994); Delaney, T. P. et al, Proc. Natl. Acad. Sci. USA 92:6602-6606 (1995); Glazebrook, J. et al., Genetics 143:973-982 (1996); Shah, J. et al., Mol. Plant Microbe Interact. 10:69-78 (1997)). NPR1 expression levels become elevated upon induction by SA, INA, BTH, or pathogen infection Cao, H. et al., Cell 88:57-63 (1997). Arabidopsis npr1/nim1 mutants are impaired in their ability to induce PR gene expression and mount a SAR response, even after treatment with SA or INA.

Intensive investigations have shed some light on how NPR1 mediates SAR. NPR1 contains a bipartite nuclear localization sequence and two potential protein-protein interaction domains: an ankyrin repeat domain and a BTB/POZ domain (Cao, H. et al., Cell 88:57-63 (1997); Ryals, J. et al., Plant Cell 9:425-439 (1997)). NPR1 is shown to function as a transcriptional co-activator in a TGA2-NPR1 complex after SA treatment in an in vivo transient cell assay; this function requires the BTB/POZ domain and the oxidation of NPR1 Cys-521 and Cys-529 (Rochon et al, Plant Cell 18:3670-3685 (2006)). Nuclear localization of NPR1 protein is essential for its function (Kinkema, M. et al., Plant Cell 12:2339-2350 (2000)). Without induction, NPR1 protein forms an oligomer and is excluded from the nucleus. Redox changes mediate SAR induction, causing monomeric NPR1 to emerge and accumulate in the nucleus and activate PR gene expression (Mou, Z. et al., Cell 113:1-10 (2003)).

In search for proteins that mediate NPR1 function, several groups have identified TGA family members of basic-region leucine zipper (bZIP) transcription factors, both from Arabidopsis Zhang, Y. et al., Proc. Natl. Acad. Sci. USA 96:6523-8 (1999); Despres, C. et al., Plant Cell 12:279-90 (2000) and from rice (Chern, M.-S. et al., Plant J. 27:101-113 (2001)), as NPR1 interacting proteins. The ankyrin repeats of NPR1 are necessary and sufficient for the interaction with TGA proteins but the interaction can be abolished by npr1-1 and npr1-2 mutants Zhang, Y. et al., Proc. Natl. Acad. Sci. USA 96:6523-8 (1999). The interaction between NPR1 and TGA proteins facilitates in vitro binding of the TGA proteins (Despres, C. et al., Plant Cell 12:279-90 (2000)) and recruits them in vivo (Johnson, C. et al., Plant Cell 15:1846-1858 (2003)) to the SA-responsive promoters. In vivo interaction between NPR1 and a GAL4:TGA2 fusion (GAL4 DNA-binding domain fused to TGA2) protein leads to SA-mediated gene activation in Arabidopsis (Fan, W. et al., Plant Cell 14:1377-1389 (2002)), supporting the notion that NPR1 binds in vivo to TGA2, which mediates transcriptional activation of downstream genes. The Arabidopsis triple knockout mutant tga2tga5tga6 blocks induction of PR gene expression and pathogen resistance (Zhang, Y. et al., Plant Cell 15:2647-2653 (2003)), further supporting the hypothesis that TGA proteins mediate NPR1 function. TGA2 functions as a negative regulator of PR genes before induction (Zhang, Y. et al., Plant Cell 15:2647-2653 (2003); Rochon et al., Plant Cell 18:3670-3685 (2006)). It is thought that, after induction, TGA proteins serve to anchor NPR1 to PR gene promoters to activate the genes.

In Arabidopsis, another group of NIM1/NPR1 interacting proteins were identified and named NIMIN1, NIMIN2, and NIMIN3. These three Arabidopsis proteins share very limited sequence similarity but may be structurally related (Weigel, R. R. et al., Plant Mol Biol. 46:143-160 (2001)). Weigel, R. R. et al., Plant Cell 17:1279-1291 (2005) further show that NIMIN1 is involved in modulating PR gene expression in Arabidopsis by interaction with NPR1.

In Arabidopsis, over-expression of NPR1 leads to enhanced disease resistance to both bacterial and oomycete pathogens Cao, H. et al., Proc. Natl. Acad. Sci. 95:6531-6536 (1998). In rice, over-expression of Arabidopsis NPR1 (Chern, M.-S. et al., Plant J. 27:101-113 (2001)) or the rice homologue NH1 (Chern, M. et al., Mol. Plant-Microbe Interact. 18:511-520 (2005b)) results in enhanced resistance to the pathogen Xanthomonas oryzae pv. oryzae (Xoo), strongly suggesting the presence of a related defense pathway in rice.

We have previously reported the isolation and characterization of a novel rice gene NRR (for Negative Regulator of disease Resistance) (Chern, M. et al., Plant J. 43:623-635 (2005a)). Over-expression of NRR in rice leads to super-susceptibility to Xoo, impairing both basal and Xa21-mediated resistance. NRR interacts with both the Arabidopsis NPR1 protein and the rice NH1 protein. NRR shows limited similarity to the Arabidopsis NIMIN2 protein, only in the NPR1 interaction domain and a short EAR (ERF-associated amphiphilic repression; Ohta, M. et al., Plant Cell 13:1959-1968 (2001)) motif-like sequence (LDLNxxP) near the C-terminus. However, the function of the Arabidopsis NIMIN2 has not been demonstrated.

### BRIEF SUMMARY OF THE INVENTION

The present invention provides plants with enhanced disease resistance. In some embodiments, the plants comprise a heterologous expression cassette, the expression cassette comprising a promoter operably linked to a polynucleotide encoding a polypeptide, the polypeptide comprising:
SEQ ID NO:1, SEQ ID NO:2, SEQ ID NO:3, or SEQ ID NO:4; and
a transcriptional activation domain, wherein:
the polypeptide lacks an LDLNXXP sequence; and
wherein the plant has enhanced disease resistance compared to a plant lacking the expression cassette.

In some embodiments, the plant is a rice, wheat, tobacco or soybean plant.

In some embodiments, the polypeptide comprises SEQ ID NO:1. In some embodiments, the polypeptide comprises SEQ ID NO:2. In some embodiments, the polypeptide comprises SEQ ID NO:3. In some embodiments, the polypeptide comprises SEQ ID NO:4.

In some embodiments, the transcriptional activation domain is selected from the group consisting of a VP16 activation domain, a GAL4 transcriptional activation domain, a P14 peptide transcriptional activation domain, a P15 transcriptional activation domain, a DOF1 transcriptional activation domain, a GT-2 transcriptional activation domain, a C1 transcriptional activation domain and a VP1 transcriptional activation domain. In some embodiments, the transcriptional activation domain is a plant transcriptional activation domain. In some embodiments, the transcriptional activation domain is not a B42 transcriptional activation domain.

In some embodiments, the promoter is constitutive. In some embodiments, the promoter is tissue-specific, organ-specific or inducible.

The present invention also provides isolated polynucleotide encoding a polypeptide, the polypeptide comprising:
SEQ ID NO:1, SEQ ID NO:2, SEQ ID NO:3, or SEQ ID NO:4; and
a plant transcriptional activation domain, wherein:
the polypeptide lacks an LDLNXXP sequence; and
expression of the polypeptide in a plant enhances disease resistance of the plant compared to a plant in which the protein is not expressed.

In some embodiments, the polypeptide comprises SEQ ID NO:1. In some embodiments, the polypeptide comprises SEQ ID NO:2. In some embodiments, the polypeptide comprises SEQ ID NO:3. In some embodiments, the polypeptide comprises SEQ ID NO:4.

In some embodiments, the transcriptional activation domain is selected from the group consisting of a VP16 activation domain, a GAL4 transcriptional activation domain, a P14 peptide transcriptional activation domain, a P15 transcriptional activation domain, a DOF1 transcriptional activation domain, a GT-2 transcriptional activation domain, a C1 transcriptional activation domain and a VP1 transcriptional activation domain. In some embodiments, transcriptional activation domain is a plant transcriptional activation domain. In some embodiments, transcriptional activation domain is not a B42 transcriptional activation domain.

In some embodiments, the promoter is constitutive. In some embodiments, the promoter is tissue-specific, organ-specific or inducible.

The present invention also provides expression cassettes. In some embodiments, the expression cassettes comprise a promoter operably linked to a polynucleotide encoding a polypeptide, the polypeptide comprising:
SEQ ID NO:1, SEQ ID NO:2, SEQ ID NO:3, or SEQ ID NO:4; and
a plant transcriptional activation domain, wherein:
the polypeptide lacks an LDLNXXP sequence; and
expression of the polypeptide in a plant enhances disease resistance of the plant compared to a plant in which the polypeptide is not expressed.

In some embodiments, the promoter is constitutive. In some embodiments, the promoter is tissue-specific, organ-specific or inducible. In some embodiments, the promoter is a plant promoter.

In some embodiments, the polypeptide comprises SEQ ID NO:1. In some embodiments, the polypeptide comprises SEQ ID NO:2. In some embodiments, the polypeptide comprises SEQ ID NO:3. In some embodiments, the polypeptide comprises SEQ ID NO:4.

In some embodiments, the transcriptional activation domain is selected from the group consisting of a VP16 activation domain, a GAL4 transcriptional activation domain, a P14 peptide transcriptional activation domain, a P15 transcriptional activation domain, a DOF1 transcriptional activation domain, a GT-2 transcriptional activation domain, a C1 transcriptional activation domain and a VP1 transcriptional activation domain.

The present invention also provides a vector comprising the expression cassette as described above or elsewhere herein. In some embodiments, the vector is a plant expression vector.

The present invention also provides isolated host cells comprising an expression cassette as described above or elsewhere herein.

The present invention also provides methods of enhancing plant resistance to a pathogen or pathogens. In some embodiments, the methods comprise, introducing a nucleic acid comprising an expression cassette as described above or elsewhere herein into a plant; and selecting a plant with increased resistance to a pathogen or pathogens compared to resistance of a plant lacking the expression cassette.

Other embodiments of the invention are also provided as described herein.

### DEFINITIONS

“Enhanced disease resistance” refers to an increase in the ability of a plant to prevent pathogen infection or pathogen-induced symptoms. Enhanced resistance can be increased resistance relative to a particular pathogen species or genus or can be increased resistance to all pathogens (e.g., systemic acquired resistance).

The term “promoter” refers to regions or sequence located upstream and/or downstream from the start of transcription and which are involved in recognition and binding of RNA polymerase and other proteins to initiate transcription. A “plant promoter” is a promoter capable of initiating transcription in plant cells. A plant promoter can be, but does not have to be, a nucleic acid sequence originally isolated from a plant.

The term “plant” includes whole plants, shoot vegetative organs/structures (e.g. leaves, stems and tubers), roots, flowers and floral organs/structures (e.g. bracts, sepals, petals, stamens, carpels, anthers and ovules), seed (including embryo, endosperm, and seed coat) and fruit (the mature ovary), plant tissue (e.g. vascular tissue, ground tissue, and the like) and cells (e.g. guard cells, egg cells, trichomes and the like), and progeny of same. The class of plants that can be used in the method of the invention is generally as broad as the class of higher and lower plants amenable to transformation techniques, including angiosperms (monocotyledonous and dicotyledonous plants), gymnosperms, ferns, and multicellular algae. It includes plants of a variety of ploidy levels, including aneuploid, polyploid, diploid, haploid and hemizygous.

A polynucleotide sequence is “heterologous to” an organism or a second polynucleotide sequence if it originates from a foreign species, or, if from the same species, is modified from its original form. For example, a promoter operably linked to a heterologous coding sequence refers to a coding sequence from a species different from that from which the promoter was derived, or, if from the same species, a coding sequence which is not naturally associated with the promoter (e.g. a genetically engineered coding sequence or an allele from a different ecotype or variety).

“Recombinant” refers to a human manipulated polynucleotide or a copy or complement of a human manipulated polynucleotide. For instance, a recombinant expression cassette comprising a promoter operably linked to a second polynucleotide may include a promoter that is heterologous to the second polynucleotide as the result of human manipulation (e.g., by methods described in Sambrook et al., Molecular Cloning—A Laboratory Manual, Cold Spring Harbor Laboratory, Cold Spring Harbor, N.Y., (1989) or Current Protocols in Molecular Biology Volumes 1-3, John Wiley & Sons, Inc. (1994-1998)). In another example, a recombinant expression cassette may comprise polynucleotides combined in such a way that the polynucleotides are extremely unlikely to be found in nature. For instance, human manipulated restriction sites or plasmid vector sequences may flank or separate the promoter from the second polynucleotide. One of skill will recognize that polynucleotides can be manipulated in many ways and are not limited to the examples above.

A polynucleotide “exogenous to” an individual plant is a polynucleotide which is introduced into the plant by any means other than by a sexual cross. Examples of means by which this can be accomplished are described below, and include Agrobracterium-mediated transformation, biolistic methods, electroporation, and the like. Such a plant containing the exogenous nucleic acid is referred to here as a T₁ (e.g. in Arabidopsis by vacuum infiltration) or R₀ (for plants regenerated from transformed cells in vitro) generation transgenic plant. Transgenic plants that arise from sexual cross or by selfing are descendants of such a plant.

“Pathogens” include, but are not limited to, viruses, bacteria, nematodes, fungi or insects (see, e.g., Agrios, Plant Pathology (Academic Press, San Diego, Calif. (1988)).

The phrase “nucleic acid” or “polynucleotide sequence” refers to a single or double-stranded polymer of deoxyribonucleotide or ribonucleotide bases read from the 5′ to the 3′ end. Nucleic acids may also include modified nucleotides that permit correct read through by a polymerase and do not significantly alter expression of a polypeptide encoded by that nucleic acid.

The phrase “nucleic acid sequence encoding” refers to a nucleic acid which directs the expression of a specific protein or peptide. The nucleic acid sequences include both the DNA strand sequence that is transcribed into RNA and the RNA sequence that is translated into protein. The nucleic acid sequences include both the full length nucleic acid sequences as well as non-full length sequences derived from the full length sequences. It should be further understood that the sequence includes the degenerate codons of the native sequence or sequences which may be introduced to provide codon preference in a specific host cell.

The phrase “host cell” refers to a cell from any organism. Preferred host cells are derived from plants, bacteria, yeast, fungi, insects or other animals. Methods for introducing polynucleotide sequences into various types of host cells are well known in the art.

An “expression cassette” refers to a nucleic acid construct, which when introduced into a host cell (e.g., a plant cell), results in transcription and/or translation of a RNA or polypeptide, respectively.

Two nucleic acid sequences or polypeptides are said to be “identical” if the sequence of nucleotides or amino acid residues, respectively, in the two sequences is the same when aligned for maximum correspondence as described below. The term “complementary to” is used herein to mean that the sequence is complementary to all or a portion of a reference polynucleotide sequence.

One example of algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

“Percentage of sequence identity” is determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The term “substantial identity” of polynucleotide sequences means that a polynucleotide comprises a sequence that has at least 25% sequence identity. Alternatively, percent identity can be any integer from 25% to 100%. More preferred embodiments include at least: 25%, 30%, 35%, 40%, 45%, 50%, 55%, 60%, 65%, 70%, 75%, 80%, 85%, 90%, 95%, or 99% compared to a reference sequence using the programs described herein; preferably BLAST using standard parameters, as described below. One of skill will recognize that the percent identity values above can be appropriately adjusted to determine corresponding identity of proteins encoded by two nucleotide sequences by taking into account codon degeneracy, amino acid similarity, reading frame positioning and the like. Substantial identity of amino acid sequences for these purposes normally means sequence identity of at least 40%. Preferred percent identity of polypeptides can be any integer from 40% to 100%. More preferred embodiments include at least 40%, 45%, 50%, 55%, 60%, 65%, 70%, 75%, 80%, 85%, 90%, 95%, or 99%. Most preferred embodiments include 61%, 62%, 63%, 64%, 65%, 66%, 67%, 68%, 69%, 70%, 71%, 72%, 73%, 74% and 75%. In some embodiments, polypeptides that are “substantially similar” share sequences as noted above except that residue positions which are not identical may differ by conservative amino acid changes. Accordingly, the present invention provides sequences substantially identical to SEQ ID NO:1, SEQ ID NO:2, SEQ ID NO:3 and SEQ ID NO:4. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, aspartic acid-glutamic acid, and asparagine-glutamine.

### BRIEF DESCRIPTION OF THE DRAWINGS

FIG. 1. RNA blot analysis of PR gene expression after INA induction. Wild type (WT) Arabidopsis, npr1-1 mutant, and transgenic Arabidopsis (NRR-9 and NRR-12) carrying 35S-NRR were treated with 0.65 mM INA. Two days later, tissues were collected and total RNA extracted from these samples. Ten pg of total RNA was loaded in each lane. The RNA blotted on a nitrocellulose membrane was probed with PR-1, PR-5, NRR, and 25S rRNA sequentially.

FIG. 2. Symptoms of infected Arabidopsis and growth curves of P. syringae.

(A) Leaf samples were taken 2 days after infection with Pst at a concentration 10⁶ cfu/mL. (B) Arabidopsis plants were infected with Pst at 10⁵ cfu/mL two days after treatment with 0.65 mM INA. Leaf samples were taken one week after infection. (C) Growth curves of Pst. Arabidopsis leaves were syringe-infiltrated with Pst at 10⁵ cfu/mL. For each sample, three leaves were pooled to extract Pst and the colony number was normalized to the weight of the leaves. Each data point represents the average and standard deviation of four samples.

FIG. 3. Gene expression from the BG2 promoter after induction.

(A) A schematic graph of the NRR protein depicting the locations of point mutations EK, FG, and LG. (B) After selection on medium containing hygromycin, 18 independent T1 transgenic Arabidopsis carrying either wild type NRR, EK, FG, or LG, were transferred to plates containing INA to induce expression from the BG2 promoter. GUS enzyme activity was assayed for each plant three days later. Three untransformed BG2 (WT) and three npr1-1 plants (in BG2 background) were also assayed for comparison. Each dot represents the GUS activity of one plant. (C) Progeny of two lines from each construct were selected for the GUS assay, along with the WT and npr1-1 controls. Five plants of each line were assayed individually after INA induction. Each bar represents the average and standard deviation of five plants.

FIG. 4. Growth curves of P. syringae and bacterial speck symptoms of Arabidopsis carrying wild type NRR and NRR mutants

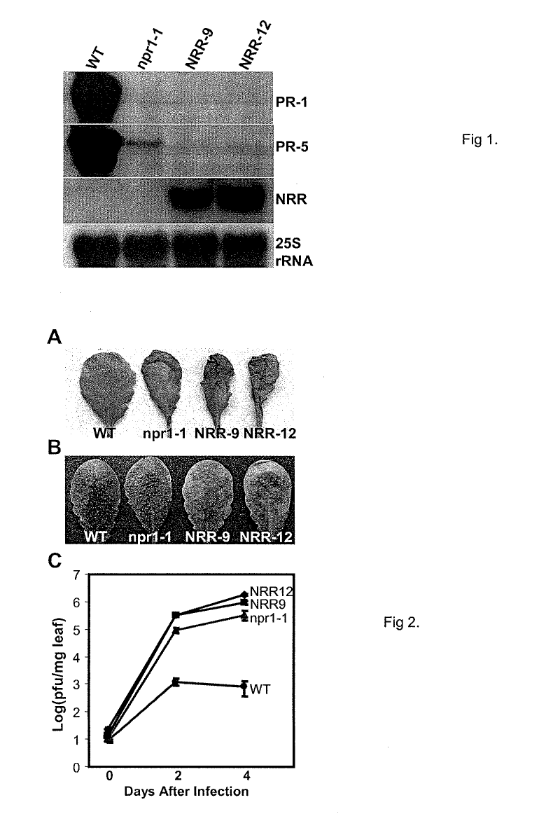

(A) Growth curves of Pst. The inoculation and growth curves were done the same way as described in FIG. 2 (B) and (C). (B) Bacterial speck symptoms of WT Arabidopsis, npr1-1, and Arabidopsis carrying wild type NRR, mutants EK, FG, and LG, after infection with Pst. (C) Western blot analysis. Equal amount (200 μg) of protein extracted from WT Arabidopsis, Arabidopsis carrying wild type NRR, mutants EK, FG, and LG, was loaded, run on a gel, and blotted to a nitrocellulose membrane. The membrane was probed with an antiserum raised against the C-terminal 83 amino acids of NRR, excluding the NI domain where the three mutations are located.

FIG. 5. PR gene expression after induction and P. syringae growth curves in NIAD plants.

(A) GUS activity driven by the BG2 promoter after INA induction. Along with the WT, 5 lines of NIAD transgenic Arabidopsis were assayed for GUS activity expressed from the BG2-Gus reporter gene after INA induction to induce PR gene expression. Each bar represents the average and standard deviation of three plants. (B) Growth curves of Pst. Arabidopsis plants were sprayed with 0.1 mM INA 24 hours before syringe-infiltration with Pst at 10⁵ cfu/mL. Growth curves were done similarly as described in FIG. 2(C). Each data point represents the average and standard deviation of three samples.

FIG. 6 shows alignment of various NRR orthologs.

FIG. 7 shows alignment of a portion of the rice NRR protein that includes the NPR1-binding domain, with NRR orthologs from other plants. The rice sequence is always the top sequence shown.

### DETAILED DESCRIPTION

### I. Introduction

As described in the Example, the inventors have surprisingly discovered that fusions of the NPR1-binding domain of NRR coupled with a transcriptional activation domain, and expressed in plants results in plants with enhanced disease resistance. Generally, the fusion proteins of the invention will lack the C-terminal portion of the native NRR protein, for example, such that the native transcriptional repression domain is either absent or inactivated. In some embodiments, the fusion protein lacks an LDLNxxP domain (where ‘x’ is any amino acid) such as is found in the C-terminal region of native NRR proteins.

II. NRR Proteins and NPR1-Binding Domains thereof

NRR proteins were first reported in Chern, M. et al., Plant J. 43:623-635 (2005), which described the rice NRR ortholog. NRR was identified in a two-hybrid system based on NRR's ability to bind to NPR1 and NPR1's rice ortholog, NH1. See, e.g., U.S. Pat. No. 6,995,306. As described in the Examples, a minimal NPR1-binding domain of NRR is present at amino acids 28-52 of the Arabidopsis NRR protein. FIGS. 6-7 set forth alignment of the NPR1 binding regions of several NRR orthologs, thereby establishing various consensus sequences representing conserved amino acid sequences within the NPR1-binding domain. Those of skill in the art will appreciate that variants of these consensus sequences can be obtained by either identifying additional NRR ortholog sequences from other plants, or by generating directed or random mutations in the sequences. NPR1-binding ability can be determined by any number of methods, including using the two-hybrid system, for example as described in U.S. Pat. No. 6,995,306. In some embodiments, the NRR NPR1-binding domain is selected from a sequence identical to, or substantially identical to, SEQ ID NO:1, SEQ ID NO:2, SEQ ID NO:3, SEQ ID NO:4, SEQ ID NO:5, SEQ ID NO:6, SEQ ID NO:7, or SEQ ID NO:8. In some embodiments, the NPR1-binding domain comprises the Arabidopsis NIMIN2 NPR1 interaction binding domain.

### III. Transcriptional Activation Domains

As described in the Example, fusion of a heterologous transcriptional activation domain to an NPR1-binding domain of NRR results in a fusion polypeptide capable of enhancing plant disease resistance. The relative position of the NPR1-binding domain and the transcriptional activation domain in the fusion protein can vary, as will be appreciated by those of skill in the art. For example, in some embodiments, the NPR1-binding domain is fused directly or indirectly (e.g., with one or more amino acids between) to the N-terminus of the transcription activation domain. In some embodiments, the NPR1-binding domain is fused directly or indirectly (e.g., with one or more amino acids between) to the C-terminus of the transcription activation domain. The fusion protein can also comprise additional sequences (e.g., tags, additional domains, an N-terminal methionine, etc.). While in some embodiments, only a minimal portion of the NRR protein that allows for NPR1 binding is used in the fusion protein, in other embodiments, additional amino acids of the endogenous NRR protein are also included in the fusion. The fusion however, will generally either lack transcription repression domains of an endogenous NRR protein completely, or such domains will be inactivated by small deletion(s), point mutation(s), or other mutations or changes.

A wide variety of transcriptional activation domains are known, including a large number with activity in plants. Those of skill in the art can readily test for transcriptional activation domain activity as is known in the art. In some embodiments, the NPR1-binding domain/transcriptional activation domain fusions are tested for activity by expressing the fusions in plants and measuring the disease resistance of the plants. In some embodiments, the transcriptional activation domain is a plant transcriptional activations domain, i.e., from a protein endogenously expressed in a plant or a modified version of such a protein. In some embodiments, the transcriptional activation domain is a viral transcriptional activation domain. In some embodiments, the transcriptional activation domain is one that is not used in a two-hybrid system, e.g., is not a B42 activation domain.

Exemplary transcriptional activation domains include, but are not limited to, VP16 activation domain (see, e.g., Silveira, et al., Plant Science 172(6): 1148-1156 (2007)), a GAL4 transcriptional activation domain (see, e.g., Estruch, et al., Nuc. Acids Res. 22(19):3983-3989 (1994)), a P14 peptide transcriptional activation domain (see, e.g., Estruch, et al., Nuc. Acids Res. 22(19):3983-3989 (1994)), a P15 transcriptional activation domain (see, e.g., Estruch, et al., Nuc. Acids Res. 22(19):3983-3989 (1994)), a DOF1 transcriptional activation domain (see, e.g., Yanagisawa, Plant and Cell Physiol. 42(8):813-822 (2001)), a GT-2 transcriptional activation domain (see, e.g., Ni, et al., Plant Cell 8(6):1041-1059 (1996)), a C1 transcriptional activation domain (see, e.g., Sainz et al., Mol. Cell Bio. 17(1):115-122 (1997)) and a VP1 transcriptional activation domain (see, e.g., Hobo et al., Proc Natl Acad Sci USA. 96(26): 15348-15353 (1999)).

### IV. Preparation of Recombinant Vectors

To use isolated sequences in the above techniques, recombinant DNA vectors suitable for transformation of plant cells are prepared. Techniques for transforming a wide variety of higher plant species are well known and described in the technical and scientific literature, e.g., Weising et al. Ann. Rev. Genet. 22:421-477 (1988). A DNA sequence coding for the desired polypeptide, for example the NPR1-binding domain/transcriptional activation domain fusions as described herein, will be combined with transcriptional and translational initiation regulatory sequences which will direct the transcription of the sequence from the gene in the intended tissues of the transformed plant.

For example, for overexpression, a plant promoter fragment may be employed which will direct expression of the gene in all tissues of a regenerated plant. Alternatively, the plant promoter may direct expression of the polynucleotide of the invention in a specific tissue (tissue-specific promoters), organ (organ-specific promoters) or may be otherwise under more precise environmental control (inducible promoters). Examples of tissue-specific promoters under developmental control include promoters that initiate transcription only in certain tissues, such as fruit, seeds, flowers, pistils, or anthers. Suitable promoters include those from genes encoding storage proteins or the lipid body membrane protein, oleosin.

If proper polypeptide expression is desired, a polyadenylation region at the 3′-end of the coding region should be included. The polyadenylation region can be derived from the natural gene, from a variety of other plant genes, or from T-DNA.

The vector comprising the sequences (e.g., promoters or coding regions) from genes of the invention will typically comprise a marker gene that confers a selectable phenotype on plant cells. For example, the marker may encode biocide resistance, particularly antibiotic resistance, such as resistance to kanamycin, G418, bleomycin, hygromycin, or herbicide resistance, such as resistance to chlorosulfuron or Basta.

Constitutive Promoters

A promoter, or an active fragment thereof, can be employed which will direct expression of a nucleic acid encoding a fusion protein of the invention, in all transformed cells or tissues, e.g. as those of a regenerated plant. Such promoters are referred to herein as “constitutive” promoters and are active under most environmental conditions and states of development or cell differentiation. Examples of constitutive promoters include those from viruses which infect plants, such as the cauliflower mosaic virus (CaMV) 35S transcription initiation region (see, e.g., Dagless Arch. Virol. 142:183-191 (1997)); the 1′- or 2′-promoter derived from T-DNA of Agrobracterium tumefaciens (see, e.g., Mengiste supra (1997); O'Grady Plant Mol. Biol. 29:99-108) (1995)); the promoter of the tobacco mosaic virus; the promoter of Figwort mosaic virus (see, e.g., Maiti Transgenic Res. 6:143-156) (1997)); actin promoters, such as the Arabidopsis actin gene promoter (see, e.g., Huang Plant Mol. Biol. 33:125-139 (1997)); alcohol dehydrogenase (Adh) gene promoters (see, e.g., Millar Plant Mol. Biol. 31:897-904 (1996)); ACT11 from Arabidopsis (Huang et al. Plant Mol. Biol. 33:125-139 (1996)), Cat3 from Arabidopsis (GenBank No. U43147, Zhong et al, Mol. Gen. Genet. 251:196-203 (1996)), the gene encoding stearoyl-acyl carrier protein desaturase from Brassica napus (Genbank No. X74782, Solocombe et al. Plant Physiol. 104:1167-1176 (1994)), GPc1 from maize (GenBank No. X15596, Martinez et al. J. Mol. Biol 208:551-565 (1989)), Gpc2 from maize (GenBank No. U45855, Manjunath et al., Plant Mol. Biol. 33:97-112 (1997)), other transcription initiation regions from various plant genes known to those of skill. See also Holtorf (1995) “Comparison of different constitutive and inducible promoters for the overexpression of transgenes in Arabidopsis thaliana,” Plant Mol Biol. 29:637-646.

Inducible Promoters

Alternatively, a plant promoter may direct expression of the nucleic acids under the influence of changing environmental conditions or developmental conditions. Examples of environmental conditions that may effect transcription by inducible promoters include anaerobic conditions, elevated temperature, drought, or the presence of light. Example of developmental conditions that may effect transcription by inducible promoters include senescence and embryogenesis. Such promoters are referred to herein as “inducible” promoters.

Exemplary inducible promoters include those promoters that are specifically induced upon infection by a virulent pathogen. Selected promoters useful in the invention are discussed in PCT application WO 99/43824, and include promoters from:
a. lipoxygenases (e.g., Peng et al, J. Biol. Chem. 269:3755-3761 (1994)),
b. peroxidases (e.g., Chittoor et al. Molec. Plant-Microbe Interact. 10:861-871 (1997)),
c. hydroxymethylglutaryl-CoA reductase,
d. phenylalanine ammonia lyase,
e. glutathione-S-transferase,
f. chitinases (e.g., Zhu et al. Mol. Gen. Genet. 226:289-296 (1991)),
g. genes involved in the plant respiratory burst (e.g., Groom et al. Plant J. 10(3):515-522 (1996)); and
h. pathogenesis-related (PR) protein promoters.

Other examples of developmental conditions include cell aging, and embryogenesis. For example, the invention incorporates the senescence inducible promoter of Arabidopsis, SAG 12, (Gan and Amasino, Science, 270:1986-1988 (1995)) and the embryogenesis related promoters of LEC1 (Lotan et al., Cell, 93:1195-205 (1998)), LEC2 (Stone et al., Proc. Natl. Acad. of Sci., 98:11806-11811 (2001)), FUS3 (Luerssen, Plant J. 15:755-764(1998)), AtSERK1 (Hecht et al. Plant Physiol 127:803-816 (2001)), AGL15 (Heck et al. Plant Cell 7:1271-1282 (1995)), and BBM (BABYBOOM). Other inducible promoters include, e.g., the drought-inducible promoter of maize (Busk supra (1997)) and the cold, drought, and high salt inducible promoter from potato (Kirch Plant Mol. Biol. 33:897-909 (1997)).

Alternatively, plant promoters which are inducible upon exposure to plant hormones, such as auxins or cytokinins, are used to express the nucleic acids of the invention. For example, the invention can use the auxin-response elements E1 promoter fragment (AuxREs) in the soybean (Glycine max L.) (Liu Plant Physiol. 115:397-407 (1997)); the auxin-responsive Arabidopsis GST6 promoter (also responsive to salicylic acid and hydrogen peroxide) (Chen Plant J. 10:955-966 (1996)); the auxin-inducible parC promoter from tobacco (Sakai 37:906-913 (1996)); a plant biotin response element (Streit Mol. Plant Microbe Interact. 10:933-937 (1997)); and, the promoter responsive to the stress hormone abscisic acid (Sheen Science 274:1900-1902 (1996)). The invention can also use the cytokinin inducible promoters of ARR5 (Brandstatter and Kieber, Plant Cell, 10: 1009-1019 (1998)), ARR6 (Brandstatter and Kieber, Plant Cell, 10:1009-1019 (1998)), ARR2 (Hwang and Sheen, Nature, 413:383-389 (2001)), the ethylene responsive promoter of ERF1 (Solano et al., Genes Dev. 12:3703-3714 (1998)), and the β-estradiol inducible promoter of XVE (Zuo et al., Plant J, 24:265-273 (2000)).

Plant promoters which are inducible upon exposure to chemicals reagents which can be applied to the plant, such as herbicides or antibiotics, are also used to express the nucleic acids of the invention. For example, the maize In2-2 promoter, activated by benzenesulfonamide herbicide safeners, can be used (De Veylder Plant Cell Physiol. 38:568-577 (1997)) as well as the promoter of the glucocorticoid receptor protein fusion inducible by dexamethasone application (Aoyama, Plant J., 11:605-612 (1997)); application of different herbicide safeners induces distinct gene expression patterns, including expression in the root, hydathodes, and the shoot apical meristem. The coding sequence of the described nucleic acids can also be under the control of, e.g., a tetracycline-inducible promoter, e.g., as described with transgenic tobacco plants containing the Avena sativa L. (oat) arginine decarboxylase gene (Masgrau Plant J. 11:465-473 (1997)); or, a salicylic acid-responsive element (Stange Plant J. 11:1315-1324 (1997)).

Tissue-Specific Promoters

Examples of tissue-specific promoters under developmental control include promoters that initiate transcription only (or primarily only) in certain tissues, such as vegetative tissues, e.g., roots, leaves or stems, or reproductive tissues, such as fruit, ovules, seeds, pollen, pistils, flowers, or any embryonic tissue.

A variety of promoters specifically active in vegetative tissues, such as leaves, stems, roots and tubers, can also be used to express the nucleic acids used in the methods of the invention. For example, promoters controlling patatin, the major storage protein of the potato tuber, can be used, e.g., Kim Plant Mol. Biol. 26:603-615 (1994); Martin Plant J. 11:53-62 (1997). The ORF13 promoter from Agrobracterium rhizogenes which exhibits high activity in roots can also be used (Hansen Mol. Gen. Genet. 254:337-343 (1997)). Other useful vegetative tissue-specific promoters include: the tarin promoter of the gene encoding a globulin from a major taro (Colocasia esculenta L. Schott) corm protein family, tarin (Bezerra Plant Mol. Biol. 28:137-144 (1995)); the curculin promoter active during taro corm development (de Castro Plant Cell 4:1549-1559 (1992)) and the promoter for the tobacco root-specific gene TobRB7, whose expression is localized to root meristem and immature central cylinder regions (Yamamoto Plant Cell 3:371-382 (1991)).

Leaf-specific promoters, such as the ribulose biphosphate carboxylase (RBCS) promoters can be used. For example, the tomato RBCS1, RBCS2 and RBCS3A genes are expressed in leaves and light-grown seedlings, only RBCS1 and RBCS2 are expressed in developing tomato fruits (Meier FEBS Lett. 415:91-95 (1997)). A ribulose bisphosphate carboxylase promoters expressed almost exclusively in mesophyll cells in leaf blades and leaf sheaths at high levels, described by Matsuoka Plant J. 6:311-319 (1994), can be used. Another leaf-specific promoter is the light harvesting chlorophyll a/b binding protein gene promoter, see, e.g., Shiina Plant Physiol. 115:477-483 (1997); Casal Plant Physiol. 116:1533-1538 (1998). The Arabidopsis thaliana myb-related gene promoter (Atmyb5) described by Li FEBS Lett. 379:117-121 (1996), is leaf-specific. The Atmyb5 promoter is expressed in developing leaf trichomes, stipules, and epidermal cells on the margins of young rosette and cauline leaves, and in immature seeds. Atmyb5 mRNA appears between fertilization and the 16-cell stage of embryo development and persists beyond the heart stage. A leaf promoter identified in maize by Busk Plant J. 11:1285-1295 (1997), can also be used.

Another class of useful vegetative tissue-specific promoters are meristematic (root tip and shoot apex) promoters. For example, the “SHOOTMERISTEMLESS” and “SCARECROW” promoters, which are active in the developing shoot or root apical meristems, described by Di Laurenzio Cell 86:423-433 (1996); and, Long Nature 379:66-69 (1996); can be used. Another useful promoter is that which controls the expression of 3-hydroxy-3-methylglutaryl coenzyme A reductase HMG2 gene, whose expression is restricted to meristematic and floral (secretory zone of the stigma, mature pollen grains, gynoecium vascular tissue, and fertilized ovules) tissues (see, e.g., Enjuto Plant Cell. 7:517-527 (1995)). Also useful are kn1-related genes from maize and other species which show meristem-specific expression, see, e.g., Granger Plant Mol. Biol. 31:373-378 (1996); Kerstetter Plant Cell 6:1877-1887 (1994); Hake Philos. Trans. R. Soc. Lond. B. Biol. Sci. 350:45-51 (1995). For example, the Arabidopsis thaliana KNAT1 or KNAT2 promoters. In the shoot apex, KNAT1 transcript is localized primarily to the shoot apical meristem; the expression of KNAT1 in the shoot meristem decreases during the floral transition and is restricted to the cortex of the inflorescence stem (see, e.g., Lincoln Plant Cell 6:1859-1876 (1994)).

One of skill will recognize that a tissue-specific promoter may drive expression of operably linked sequences in tissues other than the target tissue. Thus, as used herein a tissue-specific promoter is one that drives expression preferentially in the target tissue, but may also lead to some expression in other tissues as well.

In another embodiment, a nucleic acid described in the present invention is expressed through a transposable element. This allows for constitutive, yet periodic and infrequent expression of the constitutively active polypeptide. The invention also provides for use of tissue-specific promoters derived from viruses which can include, e.g., the tobamovirus subgenomic promoter (Kumagai Proc. Natl. Acad. Sci. USA 92:1679-1683 (1995)) the rice tungro bacilliform virus (RTBV), which replicates only in phloem cells in infected rice plants, with its promoter which drives strong phloem-specific reporter gene expression; the cassava vein mosaic virus (CVMV) promoter, with highest activity in vascular elements, in leaf mesophyll cells, and in root tips (Verdaguer Plant Mol. Biol. 31:1129-1139 (1996)).

### V. Production of Transgenic Plants

DNA constructs of the invention may be introduced into the genome of the desired plant host by a variety of conventional techniques. For example, the DNA construct may be introduced directly into the genomic DNA of the plant cell using techniques such as electroporation and microinjection of plant cell protoplasts, or the DNA constructs can be introduced directly to plant tissue using biolistic methods, such as DNA particle bombardment. Alternatively, the DNA constructs may be combined with suitable T-DNA flanking regions and introduced into a conventional Agrobracterium tumefaciens host vector. The virulence functions of the Agrobracterium tumefaciens host will direct the insertion of the construct and adjacent marker into the plant cell DNA when the cell is infected by the bacteria.

Microinjection techniques are known in the art and well described in the scientific and patent literature. The introduction of DNA constructs using polyethylene glycol precipitation is described in Paszkowski et al. Embo J. 3:2717-2722 (1984). Electroporation techniques are described in Fromm et al. Proc. Natl. Acad. Sci. USA 82:5824 (1985). Biolistic transformation techniques are described in Klein et al. Nature 327:70-73 (1987).

Agrobracterium tumefaciens-mediated transformation techniques, including disarming and use of binary vectors, are well described in the scientific literature. See, for example Horsch et al. Science 233:496-498 (1984), and Fraley et al. Proc. Natl. Acad. Sci. USA 80:4803 (1983).

Transformed plant cells which are derived by any of the above transformation techniques can be cultured to regenerate a whole plant which possesses the transformed genotype and thus the desired phenotype such as increased disease resistance compared to a control plant that was not transformed or transformed with an empty vector. Such regeneration techniques rely on manipulation of certain phytohormones in a tissue culture growth medium, typically relying on a biocide and/or herbicide marker which has been introduced together with the desired nucleotide sequences. Plant regeneration from cultured protoplasts is described in Evans et al., Protoplasts Isolation and Culture, Handbook of Plant Cell Culture, pp. 124-176, MacMillilan Publishing Company, New York, 1983; and Binding, Regeneration of Plants, Plant Protoplasts, pp. 21-73, CRC Press, Boca Raton, 1985. Regeneration can also be obtained from plant callus, explants, organs, or parts thereof. Such regeneration techniques are described generally in Klee et al. Ann. Rev. of Plant Phys. 38:467-486 (1987).

The nucleic acids and encoded polypeptides of the invention can be used to confer enhanced disease resistance on essentially any plant. Thus, the invention has use over a broad range of plants, including species from the genera Asparagus, Atropa, Avena, Brassica, Citrus, Citrullus, Capsicum, Cucumis, Cucurbita, Daucus, Fragaria, Glycine, Gossypium, Helianthus, Heterocallis, Hordeum, Hyoscyamus, Lactuca, Linum, Lolium, Lycopersicon, Malus, Manihot, Majorana, Medicago, Nicotiana, Oryza, Panieum, Pannesetum, Persea, Pisum, Pyrus, Prunus, Raphanus, Secale, Senecio, Sinapis, Solanum, Sorghum, Trigonella, Triticum, Vitis, Vigna, and, Zea.

VI. Selecting for Plants with Enhanced Resistance

Plants with enhanced resistance can be selected in many ways. One of ordinary skill in the art will recognize that the following methods are but a few of the possibilities. One method of selecting plants with enhanced resistance is to determine resistance of a plant to a specific plant pathogen. Possible pathogens include, but are not limited to, viruses, bacteria, nematodes, fungi or insects (see, e.g., Agrios, Plant Pathology (Academic Press, San Diego, Calif.) (1988)). One of skill in the art will recognize that resistance responses of plants vary depending on many factors, including what pathogen or plant is used. Generally, enhanced resistance is measured by the reduction or elimination of disease symptoms when compared to a control plant. In some cases, however, enhanced resistance can also be measured by the production of the hypersensitive response (HR) of the plant (see, e.g., Staskawicz et al. Science 268(5211): 661-7 (1995)). Plants with enhanced resistance can produce an enhanced hypersensitive response relative to control plants.

Enhanced resistance can also be determined by measuring the increased expression of a gene operably linked a defense related promoter. Measurement of such expression can be measured by quantifying the accumulation of RNA or subsequent protein product (e.g., using northern or western blot techniques, respectively (see, e.g., Sambrook et al. and Ausubel et al.). A possible alternate strategy for measuring defense gene promoter expression involves operably linking a reporter gene to the promoter. Reporter gene constructs allow for ease of measurement of expression from the promoter of interest. Examples of reporter genes include: β-gal, GUS (see, e.g., Jefferson, R. A., et al., EMBO J 6:3901-3907 (1987)), green fluorescent protein, luciferase, and others.

### Examples

The following examples are offered to illustrate, but not to limit the claimed invention.

Systemic Acquired Resistance (SAR) in plants confers lasting broad-spectrum resistance to pathogens and requires the phytohormone salicylic acid (SA). Arabidopsis NPR1/NIM1 is a key regulator of the SAR response. Studies attempting to reveal the function of NPR1 and how it mediates SA signaling include isolation of proteins that interact with NPR1. Rice NRR and Arabidopsis NIMIN1, NIMIN2, and NIMIN3 proteins are the second group of NPR1 interactors besides the TGA transcription factors. We have previously shown that over-expression of NRR in rice suppresses both basal and Xa21-mediated resistance. In order to test if NRR affects NPR1-mediated SAR, we have transformed Arabidopsis with the rice NRR gene and tested its effects on the SAR response. We find that expression of NRR in Arabidopsis results in suppression of PR gene induction by SAR inducer and resistance to pathogens. These phenotypes are even more severe than those of the npr1-1 mutant. The ability of NRR to suppress PR gene induction and disease resistance is correlated with its ability to bind to NPR1 since two point mutations, which lose the ability to bind strongly to NPR1, fail to suppress NPR1, while wild type and another point mutation, which still binds to NPR1 strongly, retain the ability to suppress. Replacing the C-terminal 79 amino acids with the VP16 activation domain turns the fusion protein into a transcriptional activator. These results indicate that NRR binds to NPR1 in vivo in a protein complex to inhibit transcriptional activation of PR genes and that NRR contains a transcription repression domain for active repression.

### Expression of Rice NRR in Arabidopsis Results in Suppression of PR Gene Expression and Impaired Resistance to Pseudomonas Syringae and Powdery Mildew

The rice NRR gene was isolated based on interaction of its gene product with the Arabidopsis NPR1 protein in yeast. We set up to test if NRR would affect NPR1-mediated SAR response in Arabidopsis, normally represented by induction of PR genes after treatment with SA or INA. We introduced a 35S-NRR construct into wild type Arabidopsis (except for carrying a BG2-Gus reporter gene [BG2=PR-2]; Cao et al., 1994) where the NRR gene expression was driven by the CaMV 35S promoter.

Many lines of 35S-NRR transgenic Arabidopsis were obtained and analyzed by a qualitative GUS histochemical staining assay. Most of them showed reduced GUS expression levels compared to the wild type control after INA pre-treatment (data not shown). Northern blot analysis was then used to assess RNA expression levels quantitatively. FIG. 1 shows RNA expression levels from RNA blotting experiments of two (NRR-9 and NRR-12) of the 35S-NRR lines along with wild type (WT) and npr1-1 controls. NRR-9 and NRR-12 show expression of NRR RNA, which is absent in WT and npr1-1. After treatment with INA (0.65 mM), PR-1 and PR-5 transcripts are highly induced whereas induction is impaired in npr1-1, except for the mild induction of PR-5. NRR-9 and NRR-12, like npr1-1, both failed to respond to INA induction. Interestingly, the levels of PR-5 expression in NRR-9 and NRR-12 after induction are even lower than in the npr1-1 mutant.

When these transgenic Arabidopsis plants were inoculated with Pseudomonas syringae pv. tomato (Pst) DC3000 at high concentration (10⁶ cfu/mL) by syringe-infiltration, infected leaves of NRR-9 and NRR-12 collapsed in two days, as shown in FIG. 2A. FIG. 2A also shows that leaves of npr1-1 partially collapsed while those of wild type showed only mild speck symptoms.

To test possible effects on SAR, these plants were sprayed with 0.65 mM INA to induce SAR and, three days afterward, inoculated with Pst DC3000 at a lower concentration (10⁵ cfu/mL) by syringe-infiltration. Leaf samples of these inoculated plants are shown in FIG. 2B, where NRR-9 and NRR-12 displayed severe bacterial speck disease symptoms a week after inoculation. The npr1-1 mutant only exhibited mild disease symptoms. On the contrary, the wild type remained little affected. FIG. 2C shows the growth curves conducted on these plants in the same experiment. For each sample, three leaves were pooled to extract Pst and the colony number was normalized to the weight of the leaves. The bacterial growth curves confirm that NRR-9 and NRR-12 are severely impaired in resistance to Pst DC3000 as they harbored several orders more Pst than the wild type. The growth curves also confirmed that NRR-9 and NRR-12 are also more susceptible to Pst than the npr1-1 mutant. T tests give p values of 0.001 and 0.0001, respectively, for NRR-9 and NRR-12 compared to the npr1-1 mutant, showing highly significant differences.

When these transgenic Arabidopsis and the wild type and npr1-1 controls were grown together in our growth chamber, they were incidentally infected by pathogens in the environment and eventually showed symptoms of powdery mildew. FIGS. 2D and 2E show that NRR-9 and NRR-12 leaves were covered with powdery mildew whereas npr1-1 was less diseased; on the contrary, wild type Arabidopsis remained relatively unaffected.

These results suggest that ectopically expressed NRR greatly suppresses normal defense responses, including SAR, in Arabidopsis, leading to hyper-susceptibility to Pst DC3000. Moreover, these plants were more severely diseased than the npr1-1 mutant, which is impaired in SAR. Consistent with these observations are the data showing that NRR-9 and NRR-12 express lower levels of PR-5 than npr1-1 after INA induction.

NRR Mutants that have Lost the Ability to Interact with NPR1 Lose the Ability to Suppress SAR in Arabidopsis

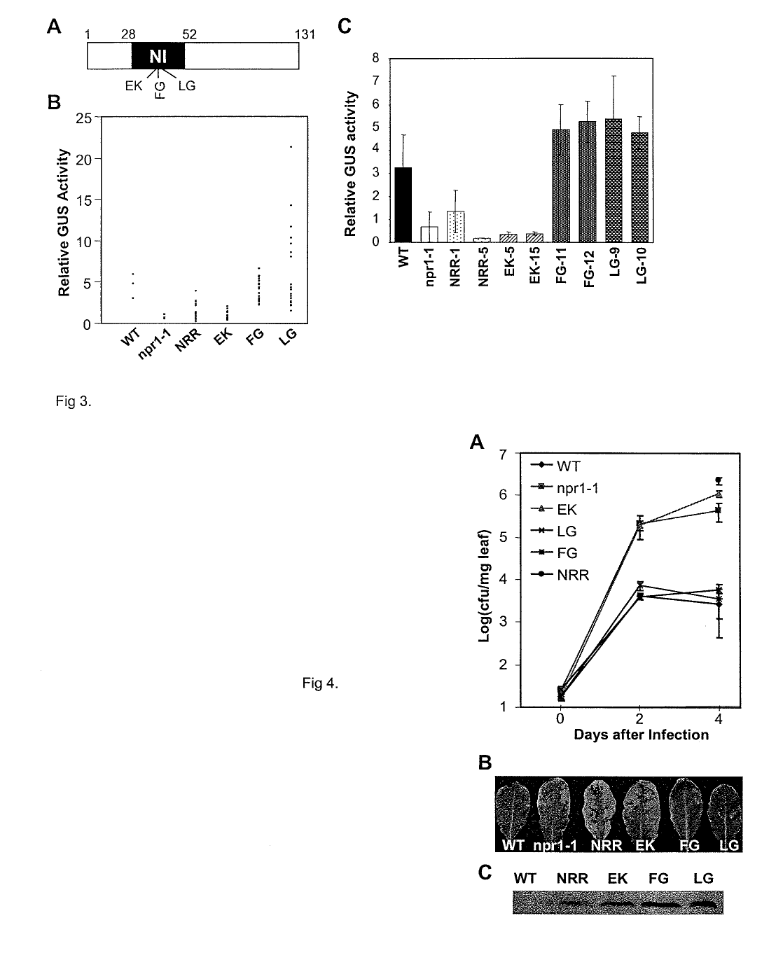

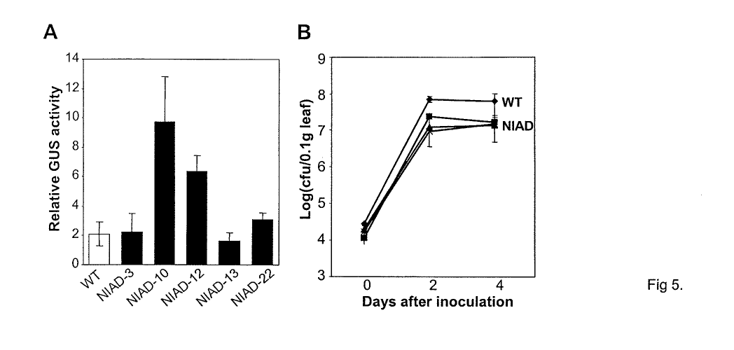

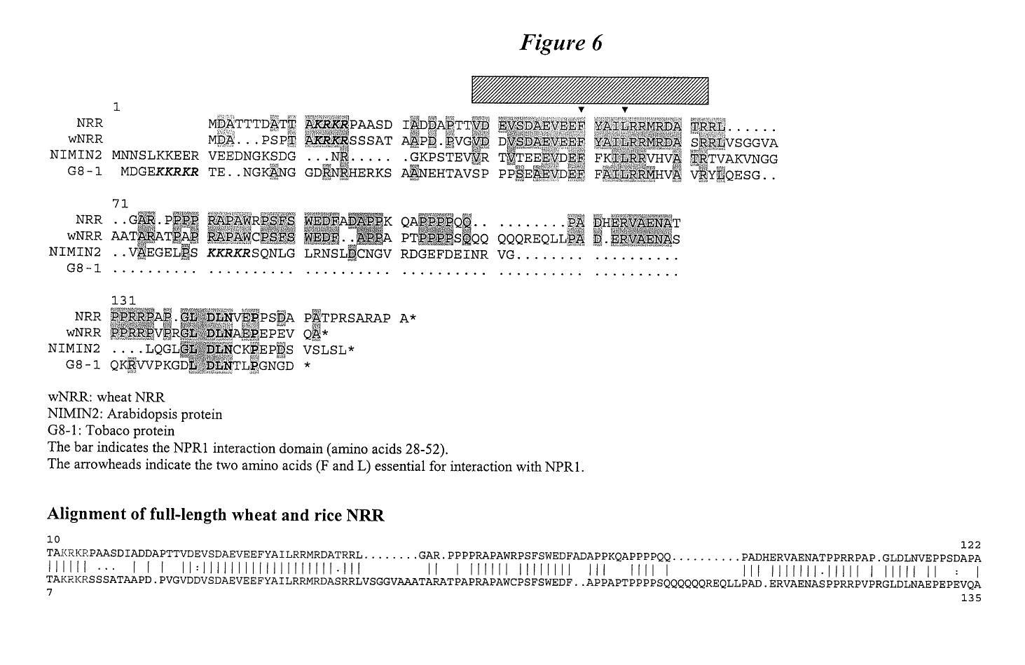

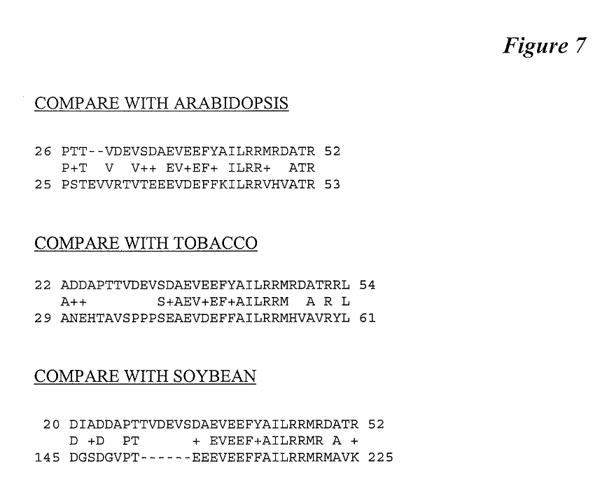

Previously, we have determined that the ability of NRR to interact with NPR1 depends on an NPR1-interacting (NI) domain comprised of 25 amino acids (amino acids 28 to 52). FIG. 3A summarizes schematically the relative locations of the NI domain and three point mutations within it. Two of the single point mutations, F40G (FG) and L44G (LG), at amino acids conserved with NIMIN2, lead to the loss of most of the ability to interact with NPR1 in yeast while E39K (EK) has little effects on interaction with NPR1. To see if the ability of NRR to suppress disease resistance is correlated with its ability to interact with NPR1, we transformed Arabidopsis with NRR mutants FG and LG under control of the 35S promoter. For comparison, transgenic Arabidopsis carrying the wild type NRR and mutant EK were also generated.

To assess the effects on expression of PR genes, we took advantage of the BG2-Gus reporter, in which the Gus gene is under control of an INA-responsive β-1,3-glucanase (BG2) promoter, in the recipient by assaying the GUS activity. After selection for the presence of the antibiotic resistance transformation marker, 18 independent T1 transgenic Arabidopsis carrying either wild type NRR, EK, FG, or LG transgene, were transferred to plates containing INA to induce expression from the BG2 promoter. Quantitative GUS enzyme activity was assayed for each plant three days afterward. For comparison, GUS activity of three untransformed BG2 (WT) and three npr1-1 plants (in BG2 background) were assayed.

The GUS activity data were plotted in FIG. 3B, where each dot represents one plant. Consistent with previous observations, the GUS activities of the majority of NRR plants shifted downward towards that of npr1-1. Plants of the EK mutants responded like the NRR pants. The FG plants responded similarly to WT. The LG plants also behaved similar to WT despite having a much broader distribution. To confirm these results, progeny of two lines from each construct were selected for the GUS assay. Five plants of each line were assayed individually after INA induction and the results are shown in FIG. 3C. FIG. 3C confirms the observations that NRR and EK transgenic Arabidopsis plants behave like npr1-1 whereas FG and LG transgenic plants responded similarly to wild type Arabidopsis. Together, these data suggest that while NRR and EK suppress PR gene induction, FG and LG, which lost the ability to interact strongly with NPR1, lose the ability. Therefore, the ability of NRR to suppress PR gene induction is correlated with its ability to interact with NPR1.

These transgenic Arabidopsis plants were also tested for response to challenge by Pst DC3000. They were syringe-infiltrated with Pst DC3000 at a concentration of 10⁵ cfu/mL after treatment with INA. FIG. 4A shows the bacterial growth curves in the different transgenic plants. Consistent with the effects on PR genes, the FG and LG mutants behaved similarly to wild type while NRR and EK responded like npr1-1. FIG. 4B shows a typical leaf from each plant 7 days after inoculation. Similarly, NRR and EK plants showed severe speck disease and npr1-1 was mildly diseased. One the contrary, the FG and LG plants, like wild type, showed few signs of the disease.

To confirm the protein was expressed in the individual transgenic plant, immunoblot analysis was performed. Protein extracted from the lines was blotted on a nitrocellulose membrane and probed with antibodies raised against a peptide corresponding to the C-terminal half of NRR. FIG. 4C shows that the mutant FG and LG proteins are at least as stable as the wild type NRR and EK proteins. Thus, loss of the suppression effects in FG and LG can not be accounted for by protein instability.

A Domain Swap Turns NRR into a Transcriptional Activator that Enhances Disease Resistance

The observation that NRR suppresses induction of PR gene induction suggests that it may contain a repression domain and act as a transcriptional repressor when bound to NPR1. One way to test this is to swap out the possible repression domain in NRR. NRR is a small protein made of 131 amino acids. Its N-terminal half contains a nuclear localization signal and the NPR1-interacting domain; its C-terminal half is proline- and alanine-rich and contains an LDLNxxP sequence, resembling the ERF-associated amphiphilic repression (EAR) motif (Ohta, M. et al., Plant Cell 13:1959-1968 (2001)), near the C-terminal end. To swap out the putative repression domain, the N-terminal first 52 amino acids of NRR was fused to a VP16 activation domain (replacing the C-terminal 79 amino acids), resulting in a fusion protein NIAD.

The NIAD construct was transformed into Arabidopsis under control of a maize ubiquitin promoter (Christensen, A. H. Transgenic Res. 5:213-218 (1996)). Progeny of 5 lines were assayed for GUS activity from the BG2-Gus reporter gene after 0.1 mM INA induction of PR gene expression. FIG. 5A shows the GUS activity of the average of three plants. Lines NIAD-10 and NIAD-12 showed much higher GUS activity than the wild type. NIAD-22 showed only slightly higher GUS activity in FIG. 5A, but when the assay was repeated with more plants, it exhibited a two-fold higher GUS activity than WT (data not shown).

These three NIAD lines (NIAD-10, NIAD-12, and NIAD-22) along with the wild type control were challenged with Pst DC3000 one day after induction INA. We chose to spread the plants with 0.1 mM INA instead of 0.65 mM INA because 0.65 mM INA strongly induces SAR and can easily mask any possible enhanced resistance responses. FIG. 5B shows the bacterial growth curves in these plants where each data point represents three repeats. The bacterial populations in all three NIAD lines are lower than in the wild type. At day 4 after inoculation, the average bacterial populations in the wild type are approximately 4- to 5-fold larger than those in the three NIAD lines. When the 9 data points of all three NIAD lines are taken into consideration, the T test gives a p value of 0.0023, demonstrating a highly significant difference between NIAD and the wild type control. Together these results show that expression of the NIAD fusion protein enhances induction of PR gene expression after INA treatment leading to higher resistance to Pst DC3000. These data also indicate that the NIAD fusion protein functions as a transcriptional activator when bound to NPR1 in Arabidopsis.

### Discussion

We have shown that expression of the rice NRR in Arabidopsis results in severe suppression of the SAR response. This suppression is dependent on the ability to interact with the NPR1 protein. These results suggest that NRR binds directly to NPR1 in vivo to inhibit the function of NPR1. Arabidopsis NPR1 is thought to act as a transcriptional co-activator as demonstrated in a transient assay system (Rochon et al., Plant Cell 18:3670-3685 (2006)). NRR contains an EAR-like motif (LDLNxxP) near its C-terminus. Thus, NRR most likely acts as a transcriptional repression by active repression. Indeed, replacing the C-terminal 79 amino acids of NRR with a VP16 activation domain turns the fusion protein NIAD into a co-activator. Expression of NIAD in Arabidopsis leads to stronger activation of the BG2 (PR-2) promoter, which is representative of PR genes, after INA induction of the SAR response. These data suggest that NIAD is present in a complex with NPR1 and TGA that binds to and activates the BG2 promoter. It therefore indicates that NRR probably forms a complex with NPR1 and TGA in vivo to act as a transcriptional repressor.

It is interesting that expression of NRR in Arabidopsis results in suppression of the INA-induced SAR response, including activation of the PR-5 gene and resistance to Pst, even more severe than the npr1-1 mutation does. Weigel et al. (2005) showed that constitutive expression of Arabidopsis NIMIN1 in Arabidopsis led to a npr1-1 like phenotype, but no phenotypes more severe than npr1-1 were reported. These results indicate that there are differences between rice NRR and Arabidopsis NIMIN1 despite their ability to bind to NPR1. Rice NRR may carry a more potent transcriptional repression domain, which may be lacking in Arabidopsis NIMIN1. How does constitutive expression of NRR suppress SAR more severely than the npr1-1 mutant? It could be because the npr1-1 mutation still carries some residual NPR1 activity while NRR completely inhibits NPR1 function to induce PR gene expression. Another possible scenario is that NRR may also inhibit components other than NPR1. It is known that there are SA-dependent but NPR1-independent pathways leading to activation of PR genes. For example, the Arabidopsis ssi mutation causes accumulation of SA, leading to constitutive expression of PR-1, PR-2, and PR-5 in the npr1-5 background (Shah, J. et al., Plant Cell 11:191-206 (1999)). In another case, sucrose is shown to increase PR-2 and PR-5 gene expression through an SA-dependent but NPR1-independent pathway (Thibaud, M.-C. et al., Plant Physio. Biochem. 42:81-88 (2004)). NRR may inhibit one of the components involved in one of these NPR1-independent pathways.

It is conceivable that NRR may interact with Arabidopsis NPR1 paralogs, such as NPR2, NPR3, and NPR4, since NRR interacts with paralogs of NH1 (rice NPR1 homolog 1) in rice (Chern, M. unpublished). However, since Arabidopsis NPR3 and NPR4 are shown to negatively regulate the SA-dependent defense pathway and PR gene expression (Zhang, Y. et al., Plant J. 48:647-656 (2006)), NRR is not likely to inhibit NPR3 and NPR4 directly. No biological function of the Arabidopsis NPR2 gene has been reported so far. It remains possible that NPR2 may have partial overlapping function with NPR1 in mediating the SA signaling pathway and that it may mediate partial activation of PR gene expression when NPR1 is no longer functional. It is possible that NRR interacts with Arabidopsis NPR2 and interferes with NPR2 function.

### Methods

### Generation of Mutant Constructs and Transgenic Arabidopsis

A 35S-C1300 vector was first created by cloning the CaMV35S promoter and a nos 3′ terminator sequence into the pCambia 1300 vector. Wild type NRR and mutants genes EK, FG, and LG were then cloned into the BamHI site behind the 35S promoter to generate 35S-NRR/C1300, 35-EK/C1300, 35S-FG/C1300, and 35S-LG/C1300. The Ubi-NIAD construct was created by cloning the NIAD gene chimera (a BamHI/KpnI fragment) into the BamHI and KpnI sites in the Ubi-C1300 vector. Generation of NRR single point mutants E39K, F40G, L44G, and chimera NIAD containing the first 52 amino acids (NI) and VP16 activation domain (AD) has been described before (Chern, M. et al., Plant J. 43:623-635 (2005a)).

To generate transgenic Arabidopsis, each of the constructs described above was used to transform Agrobacterium EHA105, which was then used to transform Arabidopsis thaliana Co1 using a dipping method (Bechtold et al., 1993). Arabidopsis seeds were germinated on MS (Murashige and Skoog) medium containing 50 μg/mL hygromycin for selection.

### SAR Induction by INA Treatment

For quantitative GUS activity measurement, the plants were grown for several days on MS basal medium agar plates containing 0.1 mM INA. The plant was ground up in GUS buffer and GUS enzyme activity assay was performed as described by Jefferson (1987). For P. syringae disease testing and RNA extraction, plants were grown on soil for several weeks and 0.1 mM or 0.65 mM INA was spread by foliar application one to three days before the assays.

RNA Blot Analysis and P. syringae Disease Testing

Total RNA was extracted from harvested tissue using the Trizol reagent (Invitrogen) according to the manufacturer's instruction. RNA was run on denaturizing agarose gels containing formaldehyde. The RNA was then transferred to a nitrocellulose membrane and hybridized to different probes. P. syringae was resuspended in 10 mM MgSO₄ solution at the desired concentration and infiltrated into each leaf by a 1-mL syringe. For P. syringae growth curves, three leaves were collected for each data point and ground up in 10 mM MgSO₄ solution to extract the bacteria. Bacteria were plated out at a series of dilution on NYGA (0.5% peptone, 0.3% yeast extract, 2% glycerol, and 1.5% agar) medium containing rifampicin (25 mg/L).

### GUS Enzyme Activity Assay

GUS enzyme activity assay was performed as described by Jefferson, R. A. Plant Mol. Biol. Rep. 5:387-405 (1987).

### Generation of Polyclonal Antibodies Against NRR

A DNA fragment corresponding to the C-terminal 83 amino acids of NRR (NRRC) was PCR-amplified with primers NRRC-pET (TTTCATATGGACGCCACCCGACGGCTC) and mn45-4 (AGGATCCACTAGTCTCGAGTTGTAATCCGTGAGCA). The PCR product was purified and digested with NdeI+BamHI and cloned in-frame into vector pET15b, predigested with NdeI and BamHI. The NRRC peptide was expressed in E. coli BL21(DE3)pLysS cells and purified with Ni-NTA agarose resins as described before. The purified NRRC peptide was used to inject rabbits to raise polyclonal antisera. The antisera were tested against E. coli protein extracts with and without NRR to confirm specificity.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A plant comprising a heterologous expression cassette, the expression cassette comprising a promoter operably linked to a polynucleotide encoding a polypeptide, the polypeptide comprising:
SEQ ID NO:1, SEQ ID NO:2, SEQ ID NO:3, or SEQ ID NO:4; and
a transcriptional activation domain, wherein:
the polypeptide lacks an LDLNXXP sequence; and
wherein the plant has enhanced disease resistance compared to a plant lacking the expression cassette.

2. The plant of claim 1, wherein the plant is a rice, wheat, tobacco or soybean plant.

3. The plant of claim 1, wherein the polypeptide comprises SEQ ID NO:1.

4. The plant of claim 1, wherein the polypeptide comprises SEQ ID NO:2.

5. The plant of claim 1, wherein the polypeptide comprises SEQ ID NO:3.

6. The plant of claim 1, wherein the polypeptide comprises SEQ ID NO:4.

7. The plant of claim 1, wherein the transcriptional activation domain is selected from the group consisting of a VP16 activation domain, a GAL4 transcriptional activation domain, a P14 peptide transcriptional activation domain, a P15 transcriptional activation domain, a DOF1 transcriptional activation domain, a GT-2 transcriptional activation domain, a C1 transcriptional activation domain and a VP1 transcriptional activation domain.

8. The plant of claim 1, wherein the promoter is constitutive.

9. The plant of claim 1, wherein the promoter is tissue-specific, organ-specific or inducible.

10. The plant of claim 1, wherein the transcriptional activation domain is a plant transcriptional activation domain.

11. The plant of claim 1, wherein the transcriptional activation domain is not a B42 transcriptional activation domain.

12. An isolated polynucleotide encoding a polypeptide, the polypeptide comprising:
SEQ ID NO:1, SEQ ID NO:2, SEQ ID NO:3, or SEQ ID NO:4; and
a plant transcriptional activation domain, wherein:
the polypeptide lacks an LDLNXXP sequence; and
expression of the polypeptide in a plant enhances disease resistance of the plant compared to a plant in which the polypeptide is not expressed.

13. An expression cassette comprising a promoter operably linked to a polynucleotide encoding a polypeptide, the polypeptide comprising:
SEQ ID NO:1, SEQ ID NO:2, SEQ ID NO:3, or SEQ ID NO:4; and
a plant transcriptional activation domain, wherein:
the polypeptide lacks an LDLNXXP sequence; and
expression of the polypeptide in a plant enhances disease resistance of the plant compared to a plant in which the polypeptide is not expressed.

14. The expression cassette of claim 13, wherein the promoter is constitutive.

15. The expression cassette of claim 13, wherein the promoter is tissue-specific, organ-specific or inducible.

16. The expression cassette of claim 13, wherein the promoter is a plant promoter.

17. A vector comprising the expression cassette of claim 13.

18. The vector of claim 17, wherein the vector is a plant expression vector.

19. An isolated host cell comprising the expression cassette of claim 13.

20. A method of enhancing plant resistance to a pathogen, the method comprising,
introducing a nucleic acid comprising the expression cassette of claim 13 into a plant; and
selecting a plant with increased resistance to a pathogen or pathogens compared to resistance of a plant lacking the expression cassette.
